(19) United States
(12) Patent Application Publication
(10) Pub. No.: US 2018/0244457 A1
(43) Pub. Date: Aug. 30, 2018

(54) ANTIOBIOTIC PACKAGE FOR HYDROPHLIC COATED MEDICAL DEVICE

(71) Applicant: FIDELIS MEDICAL LTD., Tel Aviv (IL)

(72) Inventors: David Koosman, Annandale, MN (US); Michael Ryan, Chanhassen, MN (US); Assaf Leon, Petah Tikva (IL); Eyal Sandach, Yehud (IL)

(73) Assignee: FIDELIS MEDICAL LTD., Tel Aviv (IL)

(21) Appl. No.: 15/444,349

(22) Filed: Feb. 28, 2017

(51) Int. Cl. B65D 81/22; A61B 50/30; B65B 7/28

(52) U.S. Cl. CPC B65D 81/22; A61B 2050/005; B65B 7/28; A61B 50/30

(57) ABSTRACT

A package includes a storage chamber in which a medical device is stored, a sealing member that covers and seals the storage chamber, an inlet port and an outlet port in fluid communication with the storage chamber, and an antibiotic solution introduced into the storage chamber through the inlet port and which envelopes the medical device.

### FIELD OF THE INVENTION

The present invention relates generally to packages for medical devices, and more specifically to an antibiotic package for a medical device that has a hydrophilic coating.

### BACKGROUND OF THE INVENTION

Some medical devices, such as implants or tools, have hydrophilic coatings. Hydrophilic coatings attract water molecules, creating a lubricious surface that reduces friction. For example, surgeries in which a long guidewire or catheter is snaked through the body have less impact on the patient if the device has a hydrophilic surface, because its wetness and lubricity reduce trauma and irritation at the insertion site and lessen tissue damage along the delivery path. Another example is a penile implant, in which the hydrophilic coating facilitates insertion and reduces trauma and the risk of infection.

Hydrophilic coatings release low levels of particulates, lowering the chance of an allergic in vivo reaction. The lubricity of the hydrophilic coating helps minimize bacterial colonization on the medical device, significantly reducing the chance of infection.

A problem exists in the packaging of medical devices that have hydrophilic coatings. After opening the package to use the medical device, such as in the operating room setting, an antibiotic solution must be applied to the medical device prior to use. However, if the glove of the surgeon is wet, then any surface of the medical device that comes into contact with the glove will not be covered by the antibiotic solution.

### SUMMARY OF THE INVENTION

The present invention seeks to provide an improved antibiotic package for a medical device that has a hydrophilic coating, as is described more in detail hereinbelow, which solves the abovementioned problem of the prior art.

The package includes a storage chamber in which the medical device is stored, and which may be covered and sealed by a sealing member. The package includes an inlet port and an outlet port. Antibiotic solution may be introduced into the storage chamber through the inlet port, thereby bathing the medical device with the antibiotic solution while the medical device is sealingly stored in the package. Any trapped air inside the package exits through the outlet port.

The package of the present invention saves time and completely neutralizes the risk of an area of the medical device being non-antibiotic.

There is thus provided in accordance with a non-limiting embodiment of the invention a package including a storage chamber in which a medical device is stored, a sealing member that covers and seals the storage chamber, an inlet port and an outlet port in fluid communication with the storage chamber, and an antibiotic solution introduced into the storage chamber through the inlet port and which envelopes the medical device.

There is also provided in accordance with a non-limiting embodiment of the invention a method of packaging a medical device including storing a medical device in a package that includes a storage chamber and an inlet port and an outlet port in fluid communication with the storage chamber, using a sealing member to cover and seal the storage chamber, and introducing an antibiotic solution into the storage chamber through the inlet port such that the antibiotic solution envelopes the medical device.

The outlet port allows any trapped air in the package to exit therethrough.

### BRIEF DESCRIPTION OF THE DRAWINGS

The present invention will be understood and appreciated more fully from the following detailed description taken in conjunction with the drawings in which:

FIG. 1 is a simplified pictorial illustration of a package for a medical device, constructed and operative in accordance with a non-limiting embodiment of the present invention.

### DETAILED DESCRIPTION OF EMBODIMENTS

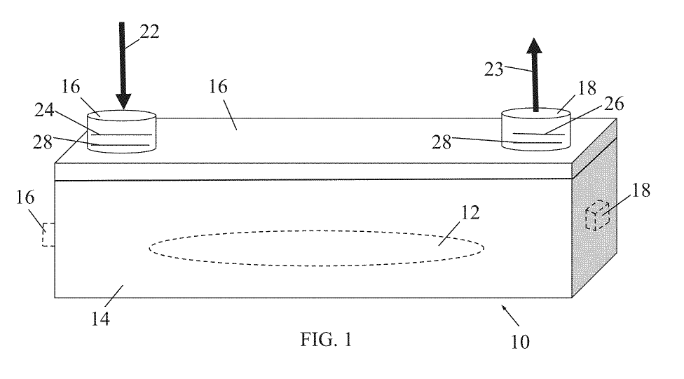

Reference is now made to FIG. 1, which illustrates a package 10 for a medical device 12, constructed and operative in accordance with a non-limiting embodiment of the present invention. The medical device 12 may have a hydrophilic coating and may include, without limitation, an implant (e.g., penile implant, spinal implant, etc.), a tool (e.g., trocar, cutter, stitcher, endoscopic tool, laparoscopic tool, etc.) and others.

The package 10 includes a storage chamber 14 in which the medical device 12 is stored. The storage chamber 14 may be of any size and shape suitable for the medical device 12. The storage chamber 14 may be covered and sealed by a sealing member 16, such as, without limitation, a sheet of TYVEK (spunbonded olefin sheet) welded, bonded or otherwise joined to the rim of storage chamber 14.

The package 10 includes an inlet port 18 and an outlet port 20, which are in fluid communication with storage chamber 14. In the illustrated embodiment, the inlet port 18 and outlet port 20 are located on top of sealing member 16. Alternatively, as shown in broken lines, inlet port 18 and outlet port 20 may be located on sides of storage chamber 14.

An antibiotic solution 22 may be introduced into the storage chamber 14 through the inlet port 18, thereby bathing the medical device 12 with the antibiotic solution 22 while the medical device 12 is sealingly stored in the package 10. Any trapped air 23 inside the package 10 exits through the outlet port 20.

Inlet port 18 may be provided with a one-way valve 24, so that the antibiotic solution 22 can enter storage chamber 14 via inlet port 18, but fluids (liquid or gas) cannot exit via inlet port 18. Outlet port 20 may be provided with a one-way valve 26, so that trapped air can exit but not the antibiotic solution 22.

The one-way valves 24 and 26 may include, without limitation, check valves, duckbill valves, umbrella valves, spring loaded ball valves, sleeve valves, diaphragm valves, reed valves, and the like.

Each of the ports may also be provided with filters 28 for ensuring that no contaminants enter the package.

What is claimed is:

1. A package comprising:
a storage chamber in which a medical device is stored;
a sealing member that covers and seals said storage chamber;
an inlet port and an outlet port in fluid communication with said storage chamber; and
an antibiotic solution introduced into said storage chamber through said inlet port and which envelopes said medical device.

2. The package according to claim 1, wherein said inlet port and said outlet port are located on top of said sealing member.

3. The package according to claim 1, wherein said inlet port and said outlet port are located on sides of said storage chamber.

4. The package according to claim 1, wherein said outlet port is configured to allow any trapped air in said package to exit therethrough.

5. The package according to claim 1, wherein said inlet port comprises a one-way valve so that said antibiotic solution can enter said storage chamber via said inlet port, but fluids cannot exit via said inlet port.

6. The package according to claim 1, wherein said outlet port comprises a one-way valve so that trapped air can exit through said outlet port but not the antibiotic solution.

7. The package according to claim 1, wherein said inlet port comprises a filter.

8. The package according to claim 1, wherein said outlet port comprises a filter.

9. The package according to claim 1, wherein said medical device has a hydrophilic coating.

10. A method of packaging a medical device comprising:
storing a medical device in a package that comprises a storage chamber and an inlet port and an outlet port in fluid communication with said storage chamber;
using a sealing member to cover and seal said storage chamber; and
introducing an antibiotic solution into said storage chamber through said inlet port such that said antibiotic solution envelopes said medical device.

11. The method according to claim 10, wherein said outlet port allows any trapped air in said package to exit therethrough.
